SEQUENCE LISTING (19 sequences)

SEQ ID NO: 1 - protein, 676 aa, Homo sapiens
MAPFLRIAFNSYELGSLQAEDEANQPFCAVKMKEALSTERGKTLVQKKPTMYPEWKSTFDAHIYEGRVIQIVLMRAAEEPVSEVTVGVSVLAERCKKNNGKAEFWLDLQPQAKVLMSVQYFLEDVDCKQSMRSEDEAKFPTMNRRGAIKQAKIHYIKNHEFIATFFGQPTFCSVCKDFVWGLNKQGYKCRQCNAAIHKKCIDKIIGRCTGTAANSRDTIFQKERFNIDMPHRFKVHNYMSPTFCDHCGSLLWGLVKQGLKCEDCGMNVHHKCREKVANLCGINQKLLAEALNQVTQRASRRSDSASSEPVGIYQGFEKKTGVAGEDMQDNSGTYGKIWEGSSKCNINNFIFHKVLGKGSFGKVLLGELKGRGEYFAIKALKKDVVLIDDDVECTMVEKRVLTLAAENPFLTHLICTFQTKDHLFFVMEFLNGGDLMYHIQDKGRFELYRATFYAAEIMCGLQFLHSKGIIYRDLKLDNVLLDRDGHIKIADFGMCKENIFGESRASTFCGTPDYIAPEILQGLKYTFSVDWWSFGVLLYEMLIGQSPFHGDDEDELFESIRVDTPHYPRWITKESKDILEKLFEREPTKRLGVTGNIKIHPFFKTINWTLLEKRRLEPPFRPKVKSPRDYSNFDQEFLNEKARLSYSDKNLIDSMDQSAFAGFSFVNPKFEHLLED

SEQ ID NO: 2 - DNA, 2028 bp, Homo sapiens
atggcgccgttcctgcgcatcgccttcaactcctatgagctgggctccctgcaggccgaggacgaggcgaaccagcccttctgtgccgtgaagatgaaggaggcgctcagcacagagcgtgggaaaacactggtgcagaagaagccgaccatgtatcctgagtggaagtcgacgttcgacgcccacatctatgaggggcgcgtcatccagattgtgctaatgcgggcagcagaggagccagtgtctgaggtgaccgtgggtgtgtcggtgctggccgagcgctgcaagaagaacaatggcaaggctgagttctggctggacctgcagcctcaggccaaggtgttgatgtctgttcagtatttcctggaggacgtggattgcaaacagtctatgcgcagtgaggacgaggccaagttcccaacgatgaaccgccgcggagccatcaaacaggccaaaatccactacatcaagaaccatgagtttatcgccaccttctttgggcaacccaccttctgttctgtgtgcaaagactttgtctggggcctcaacaagcaaggctacaaatgcaggcaatgtaacgctgccatccacaagaaatgcatcgacaagatcatcggcagatgcactggcaccgcggccaacagccgggacactatattccagaaagaacgcttcaacatcgacatgccgcaccgcttcaaggttcacaactacatgagccccaccttctgtgaccactgcggcagcctgctctggggactggtgaagcagggattaaagtgtgaagactgcggcatgaatgtgcaccataaatgccgggagaaggtggccaacctctgcggcatcaaccagaagcttttggctgaggccttgaaccaagtcacccagagagcctcccggagatcagactcagcctcctcagagcctgttgggatatatcagggtttcgagaagaagaccggagttgctggggaggacatgcaagacaacagtgggacctacggcaagatctgggagggcagcagcaagtgcaacatcaacaacttcatcttccacaaggtcctgggcaaaggcagcttcgggaaggtgctgcttggagagctgaagggcagaggagagtactttgccatcaaggccctcaagaaggatgtggtcctgatcgacgacgacgtggagtgcaccatggttgagaagcgggtgctgacacttgccgcagagaatccctttctcacccacctcatctgcaccttccagaccaaggaccacctgttctttgtgatggagttcctcaacgggggggacctgatgtaccacatccaggacaaaggccgctttgaactctaccgtgccacgttttatgccgctgagataatgtgtggactgcagtttctacacagcaagggcatcatttacagggacctcaaactggacaatgtgctgttggaccgggatggccacatcaagattgccgactttgggatgtgcaaagagaacatattcggggagagccgggccagcaccttctgcggcacccctgactatatcgcccctgagatcctacagggcctgaagtacacattctctgtggactggtggtctttcggggtccttctgtacgagatgctcattggccagtcccccttccatggtgatgatgaggatgaactcttcgagtccatccgtgtggacacgccacattatccccgctggatcaccaaggagtccaaggacatcctggagaagctctttgaaagggaaccaaccaagaggctgggagtgacgggaaacatcaaaatccaccccttcttcaagaccataaactggactctgctggaaaagcggaggttggagccacccttcaggcccaaagtgaagtcacccagagactacagtaactttgaccaggagttcctgaacgagaaggcgcgcctctcctacagcgacaagaacctcatcgactccatggaccagtctgcattcgctggcttctcctttgtgaaccccaaattcgagcacctcctggaagat

SEQ ID NO: 3 - protein, 71 aa, Artificial Sequence
  TMA
MKQAKIHYIKNHEFIATFFGQPTFCSVCKDFVWGLNKQGYKCRQCNAAIHKKCIDKIIGRCTGTAANSRDT

SEQ ID NO: 4 - DNA, 213 bp, Artificial Sequence
  TMA
atgaaacaggccaaaatccactacatcaagaaccatgagtttatcgccaccttctttgggcaacccaccttctgttctgtgtgcaaagactttgtctggggcctcaacaagcaaggctacaaatgcaggcaatgtaacgctgccatccacaagaaatgcatcgacaagatcatcggcagatgcactggcaccgcggccaacagccgggacact

SEQ ID NO: 5 - protein, 71 aa, Artificial Sequence
  TMB
MQKERFNIDMPHRFKVHNYMSPTFCDHCGSLLWGLVKQGLKCEDCGMNVHHKCREKVANLCGINQKLLAEA

SEQ ID NO: 6 - DNA, 213 bp, Artificial Sequence
  TMB
atgcagaaagaacgcttcaacatcgacatgccgcaccgcttcaaggttcacaactacatgagccccaccttctgtgaccactgcggcagcctgctctggggactggtgaagcagggattaaagtgtgaagactgcggcatgaatgtgcaccataaatgccgggagaaggtggccaacctctgcggcatcaaccagaagcttttggctgaggcc

SEQ ID NO: 7 - protein, 676 aa, Artificial Sequence
  Protein kinase C mutant, TMD
MAPFLRIAFNSYELGSLQAEDEANQPFCAVKMKEALSTERGKTLVQKKPTMYPEWKSTFDAHIYEGRVIQIVLMRAAEEPVSEVTVGVSVLAERCKKNNGKAEFWLDLQPQAKVLMSVQYFLEDVDCKQSMRSEDEAKFPTMNRRGAIKQAKIHYIKNHEFIATFFGQPTFCSVCKDFVWGLNKQGYKCRQCNAAIHKKCIDKIIGRCTGTAANSRDTIFQKERFNIDMPHRFKVHNYMSPTFCDHCGSLLWGLVKQGLKCEDCGMNVHHKCREKVANLCGINQKLLAEALNQVTQRASRRSDSASSEPVGIFQGFEKKTGVAGEDMQDNSGTYGKIWEGSSKCNINNFIFHKVLGKGSFGKVLLGELKGRGEYFAIRALKKDVVLIDDDVECTMVEKRVLTLAAENPFLTHLICTFQTKDHLFFVMEFLNGGDLMYHIQDKGRFELYRATFYAAEIMCGLQFLHSKGIIYRDLKLDNVLLDRDGHIKIADFGMCKENIFGESRASTFCGTPDYIAPEILQGLKYTFSVDWWSFGVLLYEMLIGQSPFHGDDEDELFESIRVDTPHYPRWITKESKDILEKLFEREPTKRLGVTGNIKIHPFFKTINWTLLEKRRLEPPFRPKVKSPRDYSNFDQEFLNEKARLSYSDKNLIDSMDQSAFAGFSFVNPKFEHLLED

SEQ ID NO: 8 - DNA, 2028 bp, Artificial Sequence
  Protein kinase C mutant, TMD
atggcgccgttcctgcgcatcgccttcaactcctatgagctgggctccctgcaggccgaggacgaggcgaaccagcccttctgtgccgtgaagatgaaggaggcgctcagcacagagcgtgggaaaacactggtgcagaagaagccgaccatgtatcctgagtggaagtcgacgttcgacgcccacatctatgaggggcgcgtcatccagattgtgctaatgcgggcagcagaggagccagtgtctgaggtgaccgtgggtgtgtcggtgctggccgagcgctgcaagaagaacaatggcaaggctgagttctggctggacctgcagcctcaggccaaggtgttgatgtctgttcagtatttcctggaggacgtggattgcaaacagtctatgcgcagtgaggacgaggccaagttcccaacgatgaaccgccgcggagccatcaaacaggccaaaatccactacatcaagaaccatgagtttatcgccaccttctttgggcaacccaccttctgttctgtgtgcaaagactttgtctggggcctcaacaagcaaggctacaaatgcaggcaatgtaacgctgccatccacaagaaatgcatcgacaagatcatcggcagatgcactggcaccgcggccaacagccgggacactatattccagaaagaacgcttcaacatcgacatgccgcaccgcttcaaggttcacaactacatgagccccaccttctgtgaccactgcggcagcctgctctggggactggtgaagcagggattaaagtgtgaagactgcggcatgaatgtgcaccataaatgccgggagaaggtggccaacctctgcggcatcaaccagaagcttttggctgaggccttgaaccaagtcacccagagagcctcccggagatcagactcagcctcctcagagcctgttgggatatttcagggtttcgagaagaagaccggagttgctggggaggacatgcaagacaacagtgggacctacggcaagatctgggagggcagcagcaagtgcaacatcaacaacttcatcttccacaaggtcctgggcaaaggcagcttcgggaaggtgctgcttggagagctgaagggcagaggagagtactttgccatcagggccctcaagaaggatgtggtcctgatcgacgacgacgtggagtgcaccatggttgagaagcgggtgctgacacttgccgcagagaatccctttctcacccacctcatctgcaccttccagaccaaggaccacctgttctttgtgatggagttcctcaacgggggggacctgatgtaccacatccaggacaaaggccgctttgaactctaccgtgccacgttttatgccgctgagataatgtgtggactgcagtttctacacagcaagggcatcatttacagggacctcaaactggacaatgtgctgttggaccgggatggccacatcaagattgccgactttgggatgtgcaaagagaacatattcggggagagccgggccagcaccttctgcggcacccctgactatatcgcccctgagatcctacagggcctgaagtacacattctctgtggactggtggtctttcggggtccttctgtacgagatgctcattggccagtcccccttccatggtgatgatgaggatgaactcttcgagtccatccgtgtggacacgccacattatccccgctggatcaccaaggagtccaaggacatcctggagaagctctttgaaagggaaccaaccaagaggctgggagtgacgggaaacatcaaaatccaccccttcttcaagaccataaactggactctgctggaaaagcggaggttggagccacccttcaggcccaaagtgaagtcacccagagactacagtaactttgaccaggagttcctgaacgagaaggcgcgcctctcctacagcgacaagaacctcatcgactccatggaccagtctgcattcgctggcttctcctttgtgaaccccaaattcgagcacctcctggaagat

SEQ ID NO: 9 - protein, 239 aa, Artificial Sequence
  EGFP
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 10 - DNA, 716 bp, Artificial Sequence
  EGFP
tggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 11 - protein, 225 aa, Artificial Sequence
  mRFP
MASSEDVIKEFMRFKVRMEGSVNGHEFEIEGEGEGRPYEGTQTAKLKVTKGGPLPFAWDILSPQFQYGSKAYVKHPADIPDYLKLSFPEGFKWERVMNFEDGGVVTVTQDSSLQDGEFIYKVKLRGTNFPSDGPVMQKKTMGWEASTERMYPEDGALKGEIKMRLKLKDGGHYDAEVKTTYMAKKPVQLPGAYKTDIKLDITSHNEDYTIVEQYERAEGRHSTGA

SEQ ID NO: 12 - DNA, 674 bp, Artificial Sequence
  mRFP
tggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttccagtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctccaccgagcggatgtaccccgaggacggcgccctgaagggcgagatcaagatgaggctgaagctgaaggacggcggccactacgacgccgaggtcaagaccacctacatggccaagaagcccgtgcagctgcccggcgcctacaagaccgacatcaagctggacatcacctcccacaacgaggactacaccatcgtggaacagtacgagcgcgccgagggccgccactccaccggcgcc

SEQ ID NO: 13 - DNA, 638 bp, Artificial Sequence
  Streptomyces avidinii gene for streptavidin
ccctccgtccccgccgggcaacaactagggagtatttttcgtgtctcacatgcgcaagatcgtcgttgcagccatcgccgtttccctgaccacggtctcgattacggccagcgcttcggcagacccctccaaggactcgaaggcccaggtctcggccgccgaggccggcatcaccggcacctggtacaaccagctcggctcgaccttcatcgtgaccgcgggcgccgacggcgccctgaccggaacctacgagtcggccgtcggcaacgccgagagccgctacgtcctgaccggtcgttacgacagcgccccggccaccgacggcagcggcaccgccctcggttggacggtggcctggaagaataactaccgcaacgcccactccgcgaccacgtggagcggccagtacgtcggcggcgccgaggcgaggatcaacacccagtggctgctgacctccggcaccaccgaggccaacgcctggaagtccacgctggtcggccacgacaccttcaccaaggtgaagccgtccgccgcctccatcgacgcggcgaagaaggccggcgtcaacaacggcaacccgctcgacgccgttcagcagtagtcgcgtcccggcaccggcgggtgccgggacctcggcc

SEQ ID NO: 14 - DNA, 4685 bp, Artificial Sequence
  pmRFP-C3 vector
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggtcgccaccatggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttccagtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctccaccgagcggatgtaccccgaggacggcgccctgaagggcgagatcaagatgaggctgaagctgaaggacggcggccactacgacgccgaggtcaagaccacctacatggccaagaagcccgtgcagctgcccggcgcctacaagaccgacatcaagctggacatcacctcccacaacgaggactacaccatcgtggaacagtacgagcgcgccgagggccgccactccaccggcgccctgtcagatctcgagctcaagcttcgaattctgcagtcgacggtaccgcgggcccgggatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

SEQ ID NO: 15 - DNA, 4727 bp, Artificial Sequence
  pEGFP-C3 vector
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtactcagatctcgagctcaagcttcgaattctgcagtcgacggtaccgcgggcccgggatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

SEQ ID NO: 16 - DNA, 39 bp, Artificial Sequence
  AzG-F
ggcaccggtcgccaccatggaccccatggtgagtgtgat

SEQ ID NO: 17 - DNA, 35 bp, Artificial Sequence
  AzG-R
ggcagatctgacagcttggcctgactcggcagcat

SEQ ID NO: 18 - DNA, 24 bp, Artificial Sequence
  HcR-F
gccaccggtcgccaccatggtgag

SEQ ID NO: 19 - DNA, 38 bp, Artificial Sequence
  HcR-R
gccgcggccgcttatcagttggccttctcgggcaggtc

(19) United States
(12) Patent Application Publication
(10) Pub. No.: US 2014/0335545 A1
(43) Pub. Date: Nov. 13, 2014

(54) METHOD FOR DETECTING COMPOUND-BINDING PROTEIN

(71) Applicant: KOREA BASIC SCIENCE INSTITUTE, Daejeon (KR)

(72) Inventors: Zee-Won LEE, Daejeon (KR); Soohyun Kim, Daejeon (KR); Seung II Kim, Daejeon (KR); Jung Me Hwang, Daejeon (KR)

(21) Appl. No.: 14/310,758

(22) Filed: Jun. 20, 2014

Related U.S. Application Data
(63) Continuation of application No. PCT/KR2012/011195, filed on Dec. 20, 2012.

(30) Foreign Application Priority Data
Dec. 20, 2011 (KR) 10-2011-0137868

(51) Int. Cl. G01N 33/50; G01N 33/58

(52) U.S. Cl. CPC G01N 33/5035; G01N 33/5011; G01N 33/582; G01N 33/581; G01N 2500/10; G01N 2500/02; G01N 2333/912; G01N 2333/90666
USPC 435/7.71; 435/7.1

(57) ABSTRACT

A method for detecting the interactions of biomaterials by screening a prey that interacts with a bait includes preparing a cell which expresses a first construct including a translocation module, a first labeling material, and a first medium, and a third construct including a prey and a second labeling material; introducing a second construct into the prepared cell, the second construct including a bait and a second medium binding with the first medium; allowing the prey and the bait to interact each other; and confirming the interaction between the prey and the bait by detecting intracellular distributions of the first construct and the third construct.

### CROSS REFERENCE TO RELATED APPLICATIONS

This application is a continuation of International Application No. PCT/KR2012/011195, filed on Dec. 20, 2012, which claims priority from and the benefit of Korean Patent Application No. 10-2011-0137868, filed on Dec. 20, 2011, all of which are hereby incorporated by reference for all purposes as if fully set forth herein.

### BACKGROUND

1. Field

Exemplary embodiments of the present invention relate to a method for detecting an interaction between biomaterials, and, more specifically, relate to a method for screening a prey which interacts with a bait.

2. Discussion of the Background

Growth, differentiation, migration, death, and the like of cells are mediated by macromolecular interactions such as protein-protein or protein-nucleic acid interactions. Signals from outside of cells pass through receptors located on the cellular membrane, and are transmitted to the nucleus of a cell through various biochemical reactions, where they express specific genes. This transfer of external signals into a cell is accomplished by protein interactions of several stages. For example, growth factors or cytokines bind to corresponding cell-surface receptors. This binding induces the receptors to cluster. The clustering of receptors by ligands induces clustering of the intracellular domains of the receptors, thereby causing interactions with signaling-related proteins. Through this signaling mechanism, intermediate proteins capable of transferring signals are produced by phosphorylation by protein kinases, dephosphorylation by protein phosphatases, or the like. As a result, the signals are transmitted to transcriptional activator proteins (Helden, C. H., (1995) Cell 80, 213-223). The activated transcriptional activator proteins bind to DNA and interact with basal transcriptional regulator proteins, such as RNA polymerases, to activate specific genes. Such interactions enable transcription to occur in specific tissues, in specific embryologic stages, or in response to external stimulations. Abnormal modification, inhibition, or acceleration of such interactions between specific proteins, which may be caused by intrusion of foreign matters, genetic modification of internal activator proteins, or the like, may be the cause of a disorder. Accordingly, relevant researches have been continuously conducted because substances that can regulate interactions, like the ones discussed above, may provide a way to treat the associated disorders.

The methods for analyzing the interactions of biomolecules, particularly the binding properties thereof, include traditional in vitro methods such as cross-linking, affinity chromatography, immunoprecipitation (IP), or the like. These methods require the production, isolation, and purification of a protein and are disadvantageous in that information different from the actual interaction may be obtained depending on the buffer condition in test tubes, secondary modifications of extracted proteins, or the like.

In order to correct for these drawbacks of the in vitro methods, in-cell methods such as yeast two-hybrid (Y2H), fluorescence resonance energy transfer (FRET) and bimolecular fluorescence complementation (Bi-FC) techniques have been developed. Each of these methods has advantages and disadvantages.

Y2H is currently the most widely used technique and is typically used along with immunoprecipitation. It is advantageous since large-scale screening is possible using a gene library, but is disadvantageous since investigation of membrane proteins or nuclear proteins such as transcriptase may be difficult and there may be a high probability of false positives. Besides, this method is inappropriate to find a substance capable of regulating protein-protein interactions. In the Y2H technique, the interaction between two proteins is detected based on the color change of colony to blue as X-gal compound in a medium is decomposed when β-galactosidase is expressed by the reporter gene. Since the screening technique of detecting the color change from blue back to white by a candidate substance is a negative screening, it is probable that a substance which has actually an inhibitory effect may be unnoticed. Further, since the detection itself is somewhat ambiguous, the technique is not suitable for general drug screening.

The FRET method provides good accuracy, but it is disadvantageous in that positioning of fluorescent proteins or fluorescent materials, which is required for the fluorescence resonance energy transfer to occur, is difficult, thereby having low rate of experimental success. The Bi-FC method is advantageous in that it is applicable for detecting membrane proteins or nuclear proteins. However, like the FRET method, it is disadvantageous in that relative positioning of proteins for complementary binding is difficult, thereby having low rate of success.

Therefore, various modified methods have been proposed to overcome the disadvantages of the above-described methods. However, there is a consistent need for an effective method for detecting the binding of biomaterials. Particularly, a detecting system for the detection of proteins interacting with target proteins and having a more efficient detection of regulator materials that inhibit or promote the interactions between two proteins is urgently needed.

A system has been developed in which a first construct including a protein kinase C, a first labeling material and a bait, and a second construct including a prey and a second labeling material are expressed in a cell, thereby confirming whether the expressed bait and prey interact each other, and obtained a patent for such a system (Korean Patent Registration No. 10-0948767). However, this system is usable only when both the bait and prey, of which the interaction is to be confirmed, are proteins generated in the cell, but not usable when the bait is not a protein expressed in the cell.

### SUMMARY

Exemplary embodiments of the present invention provide a system capable of separating a bait from a translocation module and a first labeling material, and introducing the separated bait.

Additional features of the invention will be set forth in the description which follows, and in part will be apparent from the description, or may be learned by practice of the invention. An exemplary embodiment of the present invention discloses a method for screening a prey that interacts with a bait, the method including, (i) creating a cell that expresses a first construct, the first construct including a translocation module, a first labeling material, and a first medium; and a third construct including a prey and a second labeling material, (ii) introducing a second construct into the created cell, the second construct including a bait and a second medium configured to bind with the first medium; (iii) reacting the prey and the bait; and (iv) detecting intracellular distributions of the first construct and the third construct to determine an interaction between the prey and the bait.

An exemplary embodiment of the present invention also discloses a cell, including: a first construct including a translocation module, a first labeling material, and a first medium; and a third construct including a prey and a second labeling material.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate exemplary embodiments of the invention, and together with the description serve to explain the principles of the invention.

FIG. 1 shows a basic conceptual view showing the detection of binding according to an exemplary embodiment of the present invention.

FIG. 2 illustrates results confirming whether a second construct can be detected by a first construct (−PMA: before treatment with signal material (PMA), +PMA: after treatment with signal material, SA: detection images of a first construct including streptavidin and RFP, Oregon green biocytin: detection images of a third construct including GFP, Merge: merging of the detection images of SA and detection images of Oregon green biocytin).

FIG. 3 shows a chemical structure of a compound-biotin (biocytin) used in FIG. 2.

FIG. 4 shows synthetic mechanisms and chemical structures of dasatinib-biotin and rapamycin-biotin.

FIG. 5 illustrates results of detecting the binding with respect to CSK, SRC, EPHA4, and RIPK2 proteins, the binding being detected by dasatinib-biotin used as a second medium (CSK: C-src tyrosine kinase, SRC: Proto-oncogene tyrosine-protein kinase Src, EPHA4: ephrin type-A receptor 4, RIPK2: Receptor-interacting serine-threonine kinase 2, (−): before treatment with signal material (PMA), +PMA: after treatment with signal material, PKC-mRFP-SA: detection images of a first construct including streptavidin and RFP, EGFP-kinase: detection images of a third construct including GFP, Merge: merging of the detection images of PKC-mRFP-SA and detection images of EGFP-kinase)

FIG. 6 illustrates results of detecting the binding with respect to CSK, SRC, and RIPK2 and charting the fluorescence change (reduction) in the cytosol (CSK: C-src tyrosine kinase, SRC: Proto-oncogene tyrosine-protein kinase Src, EPHA4: ephrin type-A receptor 4, RIPK2: Receptor-interacting serine-threonine kinase 2, A1: detection images of a first construct including RFP before treatment with PMA, A2: detection images of a third construct including GFP before treatment with PMA, A3: merging of the detection images of A1 and A2, B1: detection images of a first construct including RFP after treatment with PMA, B2: detection images of a third construct including GFP after treatment with PMA, B3: merging of the detection images of B1 and B2).

FIG. 7 illustrates results of detecting the binding with respect to CSK, SRC, and RIPK2 and charting the fluorescence change (increase) in the cytosol (CSK: C-src tyrosine kinase, SRC: Proto-oncogene tyrosine-protein kinase Src, EPHA4: ephrin type-A receptor 4, RIPK2: Receptor-interacting serine-threonine kinase 2, A1: detection images of a first construct including RFP before treatment with PMA, A2: detection images of a third construct including GFP before treatment with PMA, A3: merging of the detection images of A1 and A2, B1: detection images of a first construct including RFP after treatment with PMA, B2: detection images of a third construct including GFP after treatment with PMA, B3: merging of the detection images of B1 and B2).

FIG. 8 illustrates detection images of the binding with respect to ABL kinase family binding with dasatinib and results of immunoprecipitation (IP) for verifying the binding (CSK: C-src tyrosine kinase, ABL1: V-abl Abelson murine leukemia viral oncogene homolog 1, A1: detection images of a first construct including RFP before treatment with PMA, A2: detection images of a third construct including GFP before treatment with PMA, A3: merging of the detection images of A1 and A2, B1: detection images of a first construct including RFP after treatment with PMA, B2: detection images of a third construct including GFP after treatment with PMA, B3: merging of the detection images of B1 and B2).

FIG. 9 illustrates detection images of the binding with respect to SRC kinase family binding with dasatinib and results of immunoprecipitation (IP) for verifying the binding (SRC: Proto-oncogene tyrosine-protein kinase Src, LYN: Tyrosine-protein kinase Lyn, YES1: Proto-oncogene tyrosine-protein kinase Yes, A1: detection images of a first construct including RFP before treatment with PMA, A2: detection images of a third construct including GFP before treatment with PMA, A3: merging of the detection images of A1 and A2, B1: detection images of a first construct including RFP after treatment with PMA, B2: detection images of a third construct including GFP after treatment with PMA, B3: merging of the detection images of B1 and B2).

FIG. 10 illustrates detection images of the binding with respect to RIPK, MAP4K5, and SAPK2A proteins, which bind with dasatinib and of which functional correlation is not known, and results of immunoprecipitation (IP) for verifying the binding (RIPK2: Receptor-interacting serine-threonine kinase 2, MAP4K5: Mitogen-activated protein kinase kinase kinase kinase 5, SAPK2A: Stress Activated Protein Kinase 2a, A1: detection images of a first construct including RFP before treatment with PMA, A2: detection images of a third construct including GFP before treatment with PMA, A3: merging of the detection images of A1 and A2, B1: detection images of a first construct including RFP after treatment with PMA, B2: detection images of a third construct including GFP after treatment with PMA, B3: merging of the detection images of B1 and B2).

FIG. 11 illustrates results of verifying that phosphorylation activity of original unbiotinylated dasatinib was similar to that of biotinylated dasatinib.

FIG. 12 illustrates the binding inhibitory effect of original unbiotinylated dasatinib on CSK protein which is translocated to the membrane by dasatinib-biotin (Dasatinib: unbiotinylated dasatinib).

FIG. 13 shows (a) a schematic view of FRB and FKBP proteins of which the binding is detected by rapamycin-biotin, (b) a structure of rapamycin-biotin, and (c) the translocation to the plasma membrane and the translocation inhibitory effect by FK506 ((−) on the horizontal axis: before treatment with signal material (PMA), +PMA: after treatment with signal material, (−) on the vertical axis: rapamycin-biotin untreated group, FK506: tacrolimus, FKBP12: FK506 binding protein 12, FRB: FKBP12-rapamycin binding domain, PCK(C1A)-mRFP-SA: detection images of a first construct including streptavidin and RFP, EGFP-FRB: detection images of a third construct including GFP and FRB, TagBFP-FKBP12: detection images of a third construct including BFP and FKBP12, Merge: merging of the detection images of PCK(C1A)-mRFP-SA, EGFP-FRB, and TagBFP-FKBP12).

FIG. 14 illustrates results for verifying the inhibition of the translocation of FRB protein depending on the concentration of FK506.

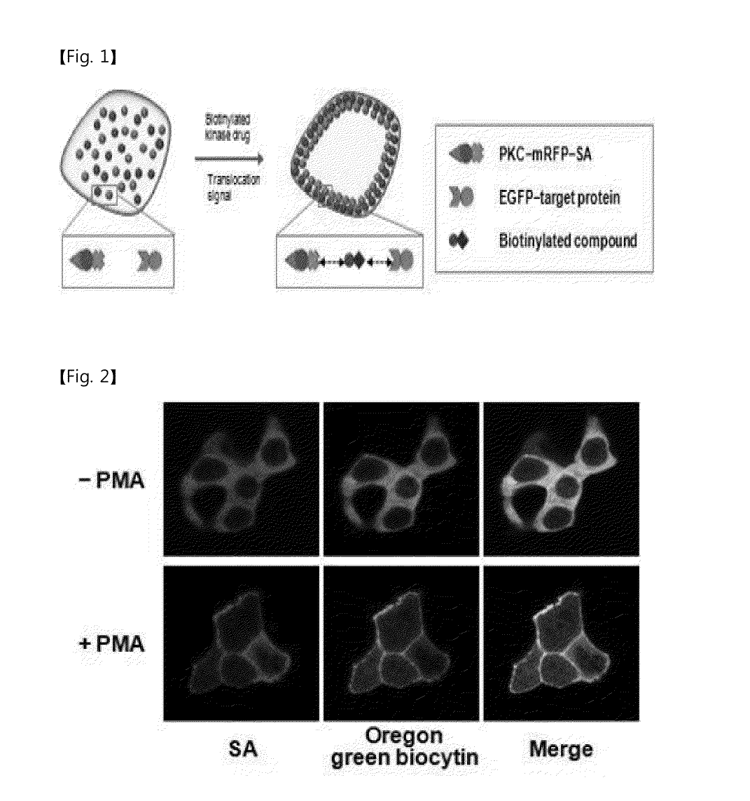

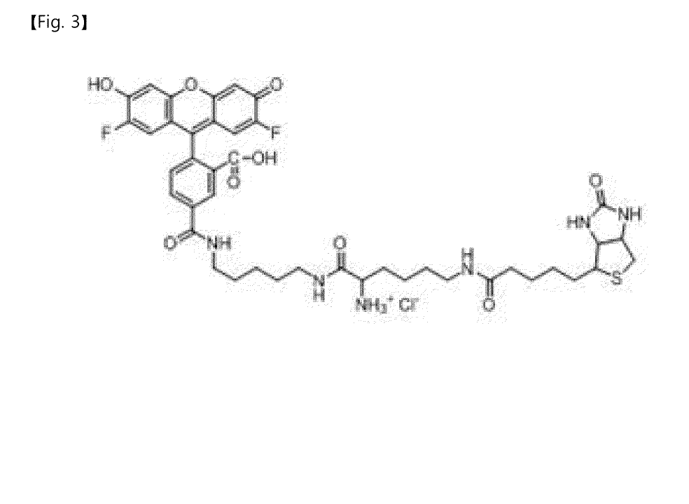

### DETAILED DESCRIPTION OF ILLUSTRATED EMBODIMENTS

The invention is described more fully hereinafter with reference to the accompanying drawings, in which embodiments of the invention are shown. This invention may, however, be embodied in many different forms and should not be construed as limited to the embodiments set forth herein. Rather, these embodiments are provided so that this disclosure is thorough, and will fully convey the scope of the invention to those skilled in the art. In the drawings, the size and relative sizes of layers and regions may be exaggerated for clarity. Like reference numerals in the drawings denote like elements.

For the direct and real-time analysis of the interaction between biomaterials, the intracellular protein translocation invoked by an external stimulation or an intrinsic signaling mechanism is used in exemplary embodiments of the present invention. In other words, the first construct is designed to include a translocation module which relocates by an external stimulation or an intrinsic signaling mechanism, a labeling material capable of tracing the translocation module, and a first medium enabling the binding of the translocation module and a bait. In addition, the second construct is designed to include a bait which is one object of the interaction, and a second medium configured to bind with the first medium to bind the bait to the first construct. In addition, the third construct is designed to include a prey interacting with the bait, and a second labeling material capable of tracing the prey. Further, in an environment into which the second construct is introduced, the first construct and the third construct are allowed to be expressed in a cell, the first construct is allowed to bind with the second construct, and the interaction between the bait and the prey is allowed to be analyzed directly and in real time in the cell.

Accordingly, exemplary embodiments of the present invention provides a method for screening a prey that interacts with a bait, the method including, (i) creating a cell that expresses a first construct, the first construct including a translocation module, a first labeling material, and a first medium; and a third construct including a prey and a second labeling material, (ii) introducing a second construct into the created cell, the second construct including a bait and a second medium configured to bind with binding with the first medium; (iii) reacting the prey and the bait; and (iv) detecting intracellular distributions of the first construct and the third construct to determine an interaction between the prey and the bait.

As used herein, “a bait” (i.e., a material of interest) and “a prey” (i.e., a target material) refer to materials that are subjected to an interaction, respectively. Each of the bait and the prey may be protein, polypeptide, small organic molecule, polysaccharide or polynucleotide, respectively. Each of the bait and prey may be protein or polypeptide. Further, they may be a natural product, synthetic compound, chemical compound, or a combination of two or more of them. For the purpose of detection or screening of an interaction, the bait may be a known material to a person conducting the detection or screening, while the prey may be an unknown material. But, without being limited thereto, the bait and the prey may be interchangeably included in the second construct or third construct.

As used herein, a first labeling material and a second labeling material refer to a material capable of generating a signal that can be distinctively detected by those skilled in the art. Examples may include fluorescent materials, ligands, light-emitting materials, microparticles, redox molecules, radioactive isotopes, or the like. As for fluorescent materials, without being limited thereto, at least one of fluorescent protein, fluorescin, isothiocyanate, rhodamine, phycoerythrin, phycocyanin, allo phycocyanin, and fluorescinisothiocyanate may be used. Among the above materials, specifically, fluorescent protein, those which are well known in the art may be used. Examples include GFP (Green Fluorescent Protein); EGFP (Enhanced Green Fluorescent Protein); RFP (Red Fluorescent Protein); mRFP (Monomeric Red Fluorescent Protein); DsRed (Discosoma sp. red fluorescent protein); CFP (Cyan Fluorescent Protein); CGFP (Cyan Green Fluorescent Protein); YFP (Yellow Fluorescent Protein); AzG (Azami Green); HcR (HcRed, Heteractis crispa red fluorescent protein); and BFP (Blue Fluorescent Protein).

Light-emitting materials may include, without being limited thereto, acridinium ester, luciferin, luciferase, or the like. Microparticles may include, without being limited thereto, colloid gold, iron, colored latex or the like. Redox molecules may include, without being limited thereto, ferrocene, ruthenium complex compounds, biologen, quinone, Ti ion, Cs ion, diimides, 1,4-benzoquinone, hydroquinone. Radioactive isotopes may include, without being limited thereto, ³H, ¹⁴C, ³²H, ³⁵S, ³⁶Cl, ⁵¹Cr, ⁵⁷Co, ⁵⁸Co, ⁵⁹Fe, ⁹⁰Y, ¹²⁵I, ¹³¹I, ¹⁸⁶Re or the like. However, any one which could be used for detecting labelled materials can be utilized as well as the above material examples.

A first labeling material and a second labeling material according to an exemplary embodiment of the present invention may be fluorescent proteins. A first labeling material and a second labeling material may be GFP; EGFP; RFP; mRFP; DsRed (Discosoma sp. red fluorescent protein); CFP (Cyan Fluorescent Protein); CGFP (Cyan Green Fluorescent Protein); YFP (Yellow Fluorescent Protein); AzG (Azami Green); HcR (HcRed, Heteractis crispa red fluorescent protein); or BFP (Blue Fluorescent Protein).

The translocation module moves the first construct to a specific region in a cell. The translocation to the specific region may be induced by an external signal or induced intrinsically. The specific region in a cell refers to an intracellular structure which is separate, discreet, and identifiable. The specific region may be membranous structures such as cell membrane, plasma membrane, and nuclear membrane; organelles such as endoplasmic reticulum, Golgi apparatus, mitochondria, and lysosome; or other specific regions in a cell.

As used herein, the translocation module may be different depending on the particular specific region in a cell. For example, it may be protein kinase C (PKC), including classical PKCs (cPKCs; PKC-alpha, PKC-beta and PKC-gamma), novel PKCs (nPKCs; PKC-delta, PKC-epsilon, PKC-eta and PKC-theta), atypical PKCs (aPKCs; PKC-zeta and PKC-lambda/iota) and their variants, or others, as known in the art.

However, all of the translocation modules have the C1 domain in common. When diacylglycerol (DAG) or phorbol ester (TPA or PMA) binds at the C1 domain, they are induced to move toward the cell membrane. A variant of PKC may be used as the translocation module in exemplary embodiments of the present invention. The variant may be one from which the internal phosphorylation active site of PKC is removed in order to minimize interference caused by an internal signaling mechanism. The translocation module may have an amino acid sequence of SEQ ID NO: 1 (PRKCD), SEQ ID NO: 3 (TMA), SEQ ID NO: 5 (TMB) or SEQ ID NO: 7 (TMD), or a nucleotide sequence of SEQ ID NO: 2 (PRKCD), SEQ ID NO: 4 (TMA), SEQ ID NO: 6 (TMB) or SEQ ID NO: 8 (TMD).

The media are used for the intracellular binding of the first construct and the second construct, and, thus, binding specificity and the binding strength therebetween of the media selected may be high. The first medium may be directly produced through gene translation and gene expression in a cell. As the second medium, a material which has a low molecular weight and, thus, can easily penetrate into a cell, may be used.

The first medium may be streptavidin and the second medium may be biotin. When the first medium is dihydrofolate reductase (DHFR), the second medium may be methotrexate (MTX), or when the first medium is a histidine polymer (His-tag), the second medium may be nickle-nitrilotriacetic acid (Ni-NTA).

The translocation of the first construct and the third construct may be detected in a cell, and may be detected in the plasma membrane. In the absence of the translocation signal, the first construct and the third construct may be detected in a cell, and may be detected in the cytosol. The translocation of the third construct to the plasma membrane is conducted by an interaction between the bait of the second construct and the prey of the third construct. Therefore, the translocation of the third construct to the plasma membrane indicates the interaction between the bait of the second construct and the prey of the third construct.

Thus, the method of exemplary embodiments of the present invention enablee real-time monitoring of direct binding or complex binding of biomolecules in a living cell through imaging, and provide the following advantages over existing techniques:

1) All bindings occurring in a living cell may be analyzed.

2) Accurate analysis may be possible because the positional change in a cell is monitored, differently from other methods where the whole cell is monitored.

3) Unlike in vitro methods, no influence from external environment occurs, because binding occurring in a living cell is monitored.

4) The binding of a bait and a prey may be monitored in real time.

5) The complex binding of a bait with multiple preys may be monitored.

6) Screening of binding of a prey to an unknown biomaterial is possible by using a mass marker library for the prey.

7) A high-throughput system can be implemented in association with a high-content screening (HCS) system.

8) Binding characteristics can be analyzed for different signaling pathways by changing the kind of external stimulation.

9) Relative quantification of the bait and the prey is possible by labeling both the first construct and second construct with labeling materials. False positive or false negative responses may be reduced because experimental errors related to the translocation of the prey in response to external stimulation or via intrinsic signaling mechanisms may be simultaneously verified.

10) The interaction between a material which is not directly produced through gene translation and gene expression in a cell and a material which is expressed in a cell can be measured.

As described above, the translocation module may move to a specific region in a cell by an external signal. Accordingly, exemplary embodiments of the inventive method for screening a prey which interacts with a bait may further include performing treatment with a signaling material. That is, the method according to an exemplary embodiment of the present invention may include (i) creating a cell that expresses a first construct, the first construct including a translocation module, a first labeling material, and a first medium; and a third construct including a prey and a second labeling material, (ii) introducing a second construct into the created cell, the second construct including a bait and a second medium configured to bind with the first medium; (iii) reacting the prey and the bait; and (iv) detecting intracellular distributions of the first construct and the third construct to determine an interaction between the prey and the bait.

The signaling material refers to a material which generates an external signal inducing the translocation of the translocation module. For example, if PKC is used as the translocation module, the signaling material may be at least one of phorbol-12-myristate 13-acetate (PMA; phorbol ester), 12-O-tetradecanoylphorbol-13-acetate (TPA), phorbol-12,13-dibutyrate (PDBu), adenosine triphosphate (ATP), tridecanoic acid, arachidonic acid, linoleic acid, DiC8, 130C937, PKC activation-related growth factors, and other PKC activating materials. The signaling material may be phorbol-12-myristate 13-acetate.

PMA may be treated at a concentration of 50 nM to 5 μM, for example, 1 μM. If the PMA concentration is below 50 nM, translocation of the PKC translocation module may be insufficient. Otherwise, if it exceeds 5 μM, excessive treatment of the chemical may lead to such undesired phenomena as cell death, signaling interference or the like.

In addition, exemplary embodiments of the present invention include a cell, including: a first construct including a translocation module, a first labeling material, and a first medium; and a third construct including a prey and a second labeling material. The translocation module, the first labeling material, the first medium, the prey, and the second labeling material are as described above.

As used herein, the cell may be a cell of an animal, a plant, a yeast, or a bacteria. Except for bacteria, the cell may be a cell which is capable of accepting the first construct introduced from outside and has well-defined boundaries of cytoplasm, nucleus, and organelles. For example, the cell may be CHO-k1 (ATCC CCL-61, Cricetulus griseus, hamster, Chinese), HEK293 (ATCC CRL-1573, Homo sapiens, human), HeLa (ATCC CCL-2, Homo sapiens, human), SH-SY5Y (ATCC CRL-2266, Homo sapiens, human), Swiss 3T3 (ATCC CCL-92, Mus musculus, mouse), 3T3-L1 (ATCC CL-173, Mus musculus, mouse), NIH/3T3 (ATCC CRL-1658, Mus musculus, mouse), L-929 (ATCC CCL-1, Mus musculus, mouse), Rat2 (ATCC CRL-1764, Rattus norvegicus, rat), RBL-2H3 (ATCC CRL-2256, Rattus norvegicus, rat), or MDCK (ATCC CCL-34, Canis familiaris). In addition, the cell may be stem cells, cells extracted from various tissues, or artificially-prepared mimic cell membrane structure.

According to exemplary embodiments of the present invention, the cell including the first construct and third construct may be prepared by molecular biological techniques known in the art. Although not limited thereto, expression vectors capable of expressing each of the first construct and the third construct, respectively, or an expression vector capable of expressing both the first construct and third construct may be introduced into a cell, so that the first construct and third construct are expressed by the expression vector(s).

To this end, as for the first construct, an expression vector including a promoter and a nucleotide encoding a first medium, a first labeling material and a translocation module, which is operably linked thereto, may be constructed. As for the third construct, an expression vector including a promoter and a nucleotide encoding a prey and a second labeling material, which is operably linked thereto, may be constructed. The two expression vectors may be simultaneously or sequentially introduced into a single cell, so that the first construct and third construct are expressed by the expression vectors. The sequence of the first medium, the first labeling material and the translocation module in the nucleotide is not set. The same is true for the nucleotide encoding the prey and the second labeling material.

As used herein, the “promoter” refers to a DNA sequence regulating the expression of nucleic acid sequence operably linked to the promoter in a specific host cell, and the term “operably linked” refers to that one nucleic acid fragment is linked to other nucleic acid fragment so that the expression thereof is affected by the other nucleic acid fragment. Additionally, the promoter may include an operator sequence for controlling transcription, a sequence encoding a suitable mRNA ribosome-binding site, and a sequence controlling the termination of transcription and translation.

The introduction of an expression vector to a cell may be performed by the transfection methods which are well known in the art, for example, calcium phosphate method, calcium chloride method, rubidium chloride method, microprojectile bombardment, electroporation, particle gun bombardment, silicon carbide whiskers, sonication, PEG-mediated fusion, microinjection, liposome-mediated method, magnetic nanoparticle-mediated method, and the like.

As for the cell, when the second construct including the bait and the second medium is introduced into the cell, the second construct binds with the first construct expressed in the cell. The second construct and the third construct bind to each other depending on whether the bait and the prey interact with each other. Therefore, the interaction between the bait and the prey can be confirmed by detecting the first labeling material and the second labeling material included in the first construct and the third construct. In this way, the interaction between a material which is not directly produced through gene translation and gene expression in the cell and a material expressed in the cell can be measured.

General recombinant DNA and molecular cloning techniques are well known in the art, and they are well described in the following references: (Sambrook, J., Fritsch, E. F. and Maniatis, T., Molecular Cloning: A Laboratory Manual, 2nd ed., Cold Spring Harbor Laboratory: Cold Spring Harbor, N.Y. (1989); Silhavy, T. J., Bennan, M. L. and Enquist, L. W., Experiments with Gene Fusions, Cold Spring Harbor Laboratory: Cold Spring Harbor, N.Y. (1984); and Ausubel, F. M. et al., Current Protocols in Molecular Biology, published by Greene Publishing Assoc. and Wiley-Interscience (1987)).

Further, exemplary embodiments of the present invention provide a kit for screening a prey interacting with a bait, the kit including a cell, including a first construct including a translocation module, a first labeling material, and a first medium, and a third construct comprising a prey and a second labeling material; and a second construct including a second medium configured to bind with a first medium; and a bait.

The kit may include a signal material for enabling the interaction between the prey and the bait. The signal material is as described above, and may be phorbol 12-myristate 13-acetate (PMA, phorbol ester).

As for the media of the kit, the first medium may be streptavidin and the second medium may be biotin. Alternatively, the first medium may be dihydrofolate reductase (DHFR) and the second medium may be methotrexate (MTX).

The kit may further include a tool and/or a reagent, which are used to detect a labeling material and are known in the art, in addition to the cell including the first construct and the third construct and the second construct including the second medium configured to bind with the first medium and the bait. The kit according to exemplary embodiments of the present invention may further include a tube for mixing each component, a well plate, an instruction manual describing how to use, or the like, according to need.

Experimental procedures, reagents, and reaction conditions that may be used in the methods according to exemplary embodiments of the present invention may be those commonly known in the art and will be obvious to those skilled in the art.

Accordingly, exemplary embodiments of the present invention provide a method for screening a prey which interacts with a bait, the method the method including, (i) creating a cell that expresses a first construct, the first construct including a translocation module, a first labeling material, and a first medium; and a third construct including a prey and a second labeling material, (ii) introducing a second construct into the created cell, the second construct including a bait and a second medium configured to bind with the first medium; (iii) reacting the prey and the bait; and (iv) detecting intracellular distributions of the first construct and the third construct to determine an interaction between the prey and the bait. Further, exemplary embodiments of the present invention provide a cell including the first construct and the third construct. Still further, exemplary embodiments of the present invention provides a screening kit including the cell and the second construct.

The methods according to exemplary embodiments of the present invention can overcome disadvantages including inaccuracy and complexity of the existing techniques for biomaterial interaction detection, and can measure the interaction between a material which is not directly produced through gene translation and gene expression in the cell and a material expressed in a cell. Thus, the methods are effective in detecting interactions of a broad range of biomaterials.

Hereinafter, embodiments of present invention will be described in detail by referring to the examples.

However, the following examples are for illustrative purposes only and are not intended to limit the scope of the present invention.

### Example 1

### Animal Cell Lines and Transformation Thereof

<1-1> Animal Cell Line and Culturing

CHO-k1 (ATCC CCL-61, Cricetulus griseus, hamster, Chinese), HEK293 (ATCC CRL-1573, Homo sapiens, human), HeLa (ATCC CCL-2, Homo sapiens, human) and SH-SY5Y (ATCC CRL-2266, Homo sapiens, human) cell lines were used. The animal cells were cultured according to the instructions of ATCC (American Type Culture Collection) for the individual cells. CHO-k1 cells were cultured by using F-12 medium, while HEK293, HeLa and SH-SY5Y cells were cultured using DMEM medium. Other culturing conditions were the same. Culturing conditions commonly shared for the said cells were as follows, however, those skilled in the art may modify the specific conditions depending on purposes. The cells were cultured in pH 7.4 medium (F-12 and DMEM) containing 25 mM HEPES, 10% fetal bovine serum (FBS, v/v), 100 units/ml penicillin and 100 μg/ml streptomycin in a 5% CO₂ incubator maintained at 37° C.

<1-2> Transformation of Cell Lines

Genes were introduced into the cells using ExGene 500 (Fermentas Life Science), a liposome-based technique. All the conditions for the gene introduction including gene concentration were pursuant to the manufacturer's instructions. More specifically, after transferring the subcultured cells to a 12-well plate with a cover slip, followed by culturing for a day, the culture medium was replaced with 0.9 ml of fresh medium. About 1 μg of the transformation sample was added to 0.1 ml of 150 mM NaCl solution. After completely mixing, 3.3 μl of ExGene reagent was added and mixed by vortexing for 15 seconds. The resultant solution was allowed to stand at room temperature for 10 minutes and then added to each well of the 12-well plate in which the cells were growing. The cells were allowed to be transformed by culturing for 18 hours.

### Example 2

### Design and Preparation of a First Construct, a Second Construct and a Third Construct

<2-1> Design and Preparation of First Construct

A first construct is a fusion construct composed of a translocation module capable of moving a protein uniformly expressed in the cytoplasm of a cell toward the plasma membrane, a first labeling material analyzable by using a microscope, and a first medium capable of binding to a second medium.

In the present example, protein kinase C was used as a translocation module, mRFP as a first labeling material, and streptavidin as a first medium.

The translocation module was cloned by PCR techniques using the pCMV-SPORT6-PRKCD vector (GenBank accession No. BC043350; purchased from Openbiosystem (http://www.openbiosystems.com/); Catalog No. EHS1001-410108-BC043350) as a template, and then inserted at the NheI/AgeI site of the pmRFP-C3 vector (mRFP; GenBank accession No. DQ903889, SEQ ID NO: 14).

Streptavidin as a first medium was cloned by PCR techniques using GenBank Acc. No. X03591 (SEQ ID NO: 13) as a template, and then inserted at the EcoRI/BamHI site of the pmRFP-C3 vector (mRFP; GenBank accession No. DQ903889, SEQ ID NO: 14).

<2-2> Design and Construction of a Second Construct

A second construct is composed of a second medium configured to bind to the first medium and a bait.

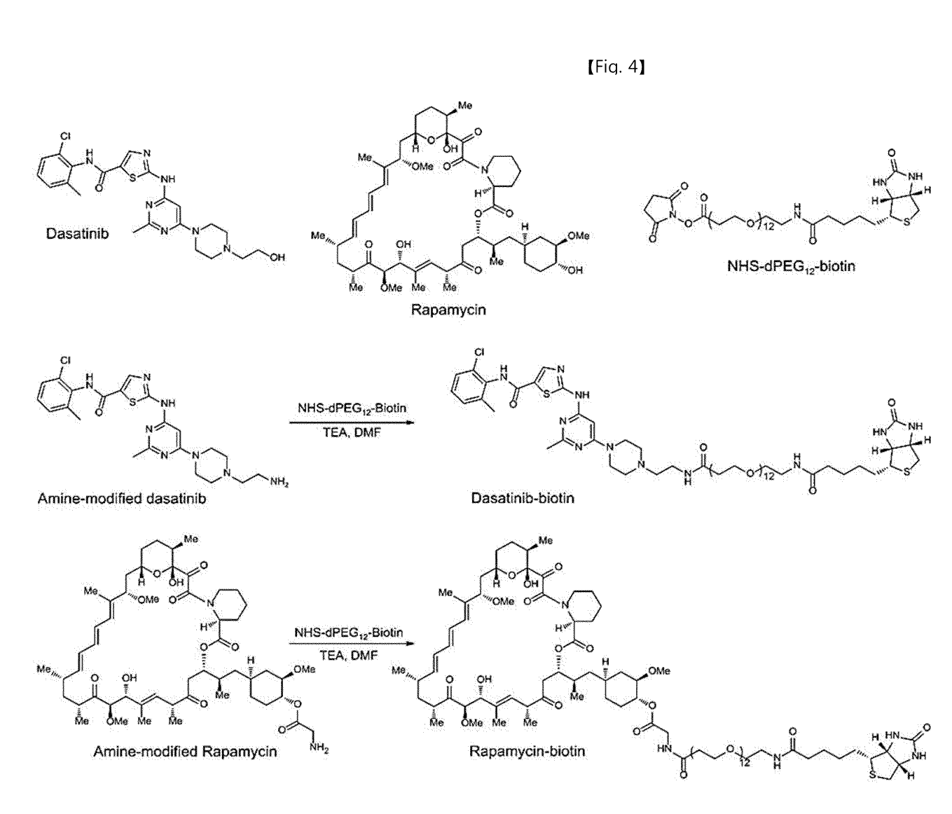

Referring to FIG. 4, biotin was used as a second medium for streptavidin, while dasatinib and rapamycin were used as a bait. Commercialized dasatinib and rapamycin were respectively substituted with an amine group, and then commercialized NHS-Dpeg12-biotin was allowed to chemically bind thereto, thereby synthesizing dasatinib-biotin and rapamycin-biotin, respectively.

<2-3> Design and Preparation of Third Construct

The third construct includes a prey which has characteristic for binding to the bait of the second construct and a labeling material for analyzing the movement of the prey. The third construct was prepared using a fluorescent material other than used in the first construct. Using green fluorescent protein (EGFP, AzG), red fluorescent protein (mRFP) and infrared fluorescent protein (HcR), the third construct was prepared by the same method described for the first construct.

When EGFP was used as the second labeling material, a pEGFP-C3 vector (Clontech; SEQ ID NO: 15) was used. When mRFP was used, a pmRFP-C3 vector was used. When AzG was used, a pAzG-C3 vector was used. When HcR was used, a pHcR-C3 vector was used. The C3 vectors had been prepared by substituting the EGFP gene sequence site of the pEGFP-C3 vector with AzG and HcR genes, as follows.

The pAzG-C3 vector was prepared as follows. PCR was carried out using a pPM-mAG1 vector (purchased from MBL, Catalog No. AM-V0203; Karasawa, S., et al. 2003, J. Biol. Chem. 278, 34167-34171) as a template and using SEQ ID NO: 16 (AzG-F: 5′-GGCACCGGTCGCCACCATGGACCCCATGGTGAGTGTGAT-3′) and SEQ ID NO: 17 (AzG-R: 5′-GGCAGATCTGACAGCTTGGCCTGACTCGGCAGCAT-3′) as primers. Then, the EGFP nucleotide sequence of the pEGFP-C3 vector was substituted at the AgeI/NotI site by the resultant PCR product.

The pHcR-C3 vector was prepared as follows. PCR was carried out using pHcRed-Tandem-N1 (purchased from Avrogen, Catalog No. FP204; Gurskaya et al., 2001, FEBS Lett. 507, 16-20.) as a template and using SEQ ID NO: 18 (HcR-F: 5′-GCCACCGGTCGCCACCATGGTGAG-3′) and SEQ ID NO: 19 (HcR-R: 5′-GCCGCGGCCGCTTATCAGTTGGCCTTCTCGGGCAGGTC-3′) as primers. Then, the EGFP nucleotide sequence of the pEGFP-C3 vector was substituted at the AgeI/NotI site by the resultant PCR product.

### Example 3

### Verification of Interactions of the First Construct, the Second Construct and the Third Construct

<3-1> Verification of Expression of Constructs and Analysis of Translocation Characteristics

Referring to FIG. 2, a cover slip containing the cells, in which the first construct and the second construct vectors had been introduced, was fixed to a perfusion chamber and mounted on the object stage of a confocal laser fluorescence microscope (Carl Zeiss LSM510). Images of the construct vectors were taken before and after external stimulation (treatment with 1 μM PMA).

As for the confocal laser fluorescence microscope, 488 nm argon laser (EGFP or AzG), 543 nm HeNe laser (mRFP), or 561 nm DPSS laser (HcR) was used to induce the excitation of the fluorescent label, and the fluorescence signal generated by each fluorescent label was filtered through the band path filter BP505-530 (EGFP or AzG), long path filter LP560 or BP560-630 (mRFP), or long path filter LP650 (HcR). Images were taken after completely removing the interference between the fluorescences.

An experiment for verifying the basic concept that a compound (Oregon green) bound to biotin as the second medium of the second construct can be translocated to the plasma membrane by streptavidin as the first medium attached to the first construct was conducted. As a result, the translocation of the second construct to the plasma membrane by the translocation of the first construct was confirmed through images.

### Example 4

### Real-Time Analysis of Intracellular Binding Using the First Construct, the Second Construct, and the Third Construct

For the verification of drug targets of dasatinib used as an anticancer drug, experiments of analyzing the binding of dasatinib with CSK, SRC, EPHA4, and RIPK2 proteins, which bind to dasatinib, were conducted.

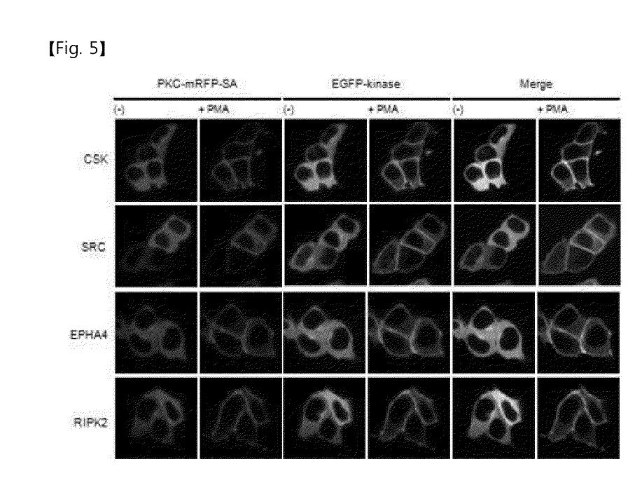

As a result, as can be seen from FIG. 5, the respective proteins (green) were simultaneously translocated to the plasma membrane by PMA as a stimulation for translocation while the first construct (red), and the second construct (dasatinib-biotin) were present.

For the verification of the intracellular binding with respect to CSK, SRC, EPHA4, and RIPK2 proteins, the first construct PKC-mRFP-streptavidin, the third construct GFP-protein expressing vector, and the second construct dasatinib-biotin were prepared as follows. The first construct PKC-mRFP-streptavidin was prepared according to the method of Example <2-1>. The second construct dasatinib-biotin was prepared according to the method of Example <2-2>. The third construct EGFP-protein was prepared by PCR amplification using a human-derived gene as a template, which is obvious to those skilled in the art, and then inserting the resultant PCR product into the pEGFP-C3 vector.

The first construct (PKC-mRFP-streptavidin) and the third construct (EGFP-protein) were introduced into CHO-k1 cells by the methods described in Example <1-2>(ExGene 500), and then allowed to be expressed in an environment into which the second construct (dasatinib-biotin) was introduced. After the 18-hour culturing and 1 uM PMA treatment for 5 minutes, the fluorescent distribution was observed, as in Example <3-1>.

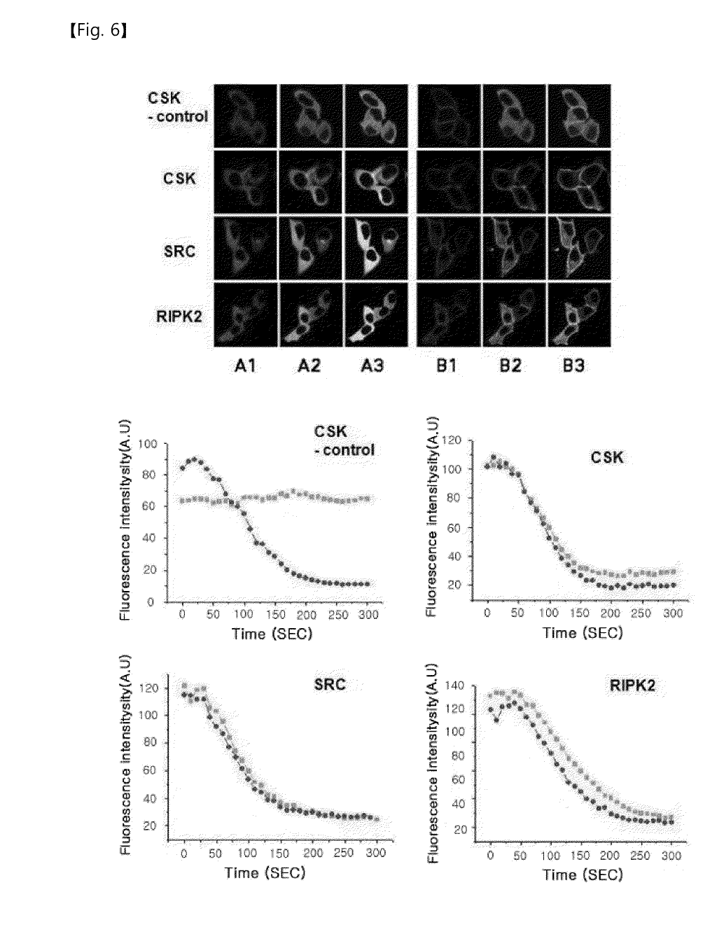

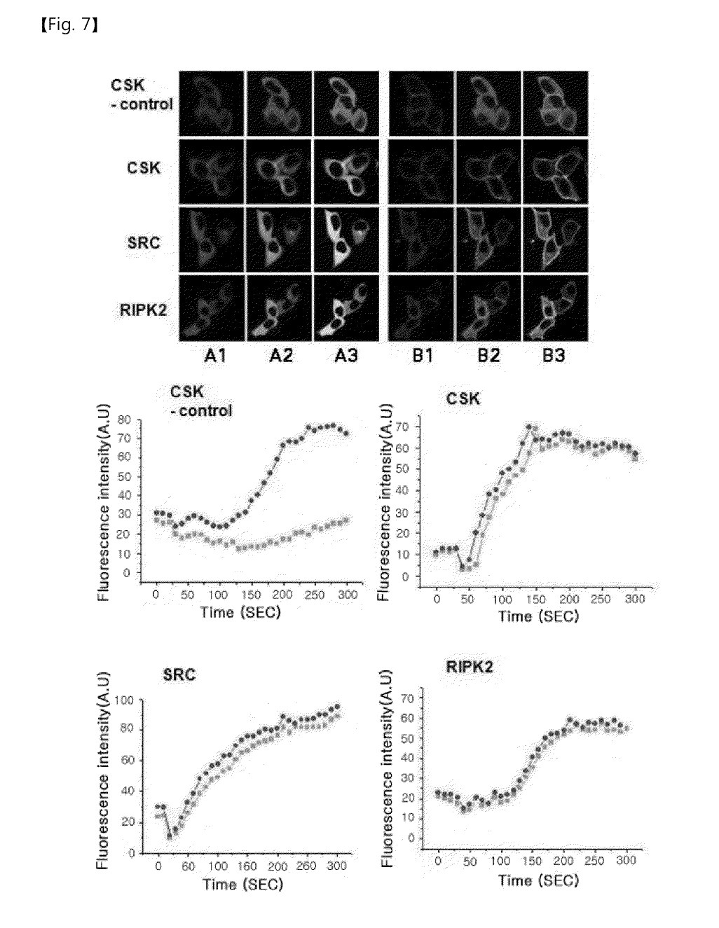

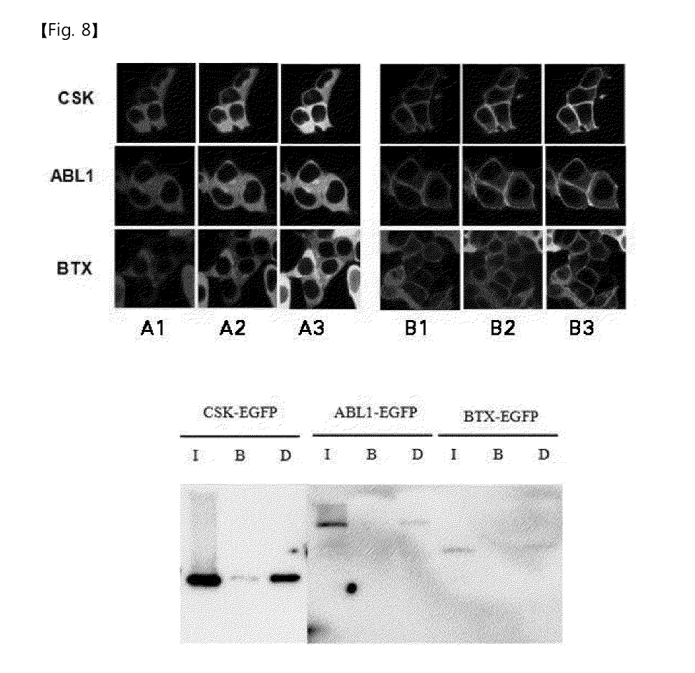

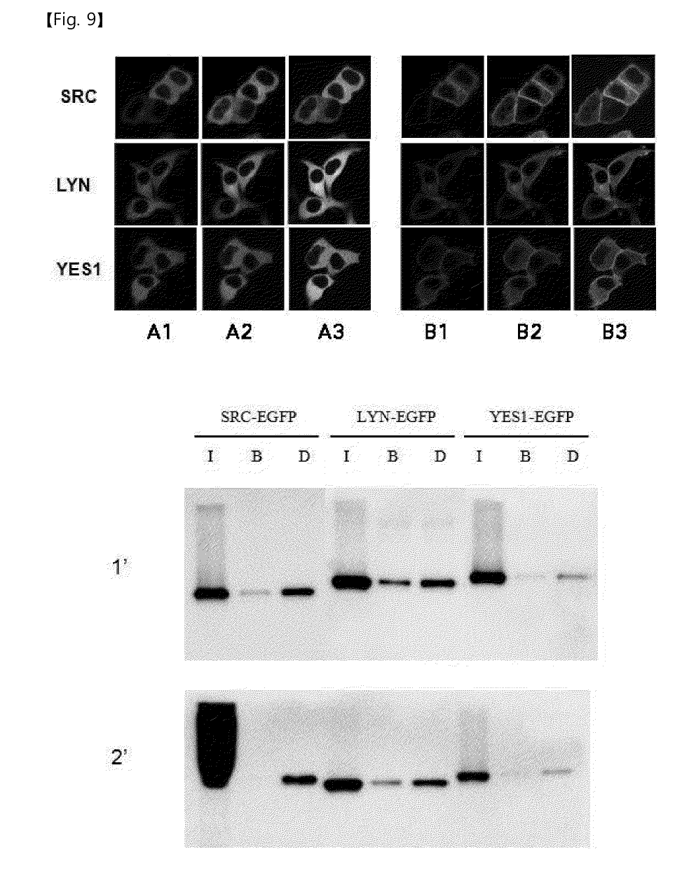

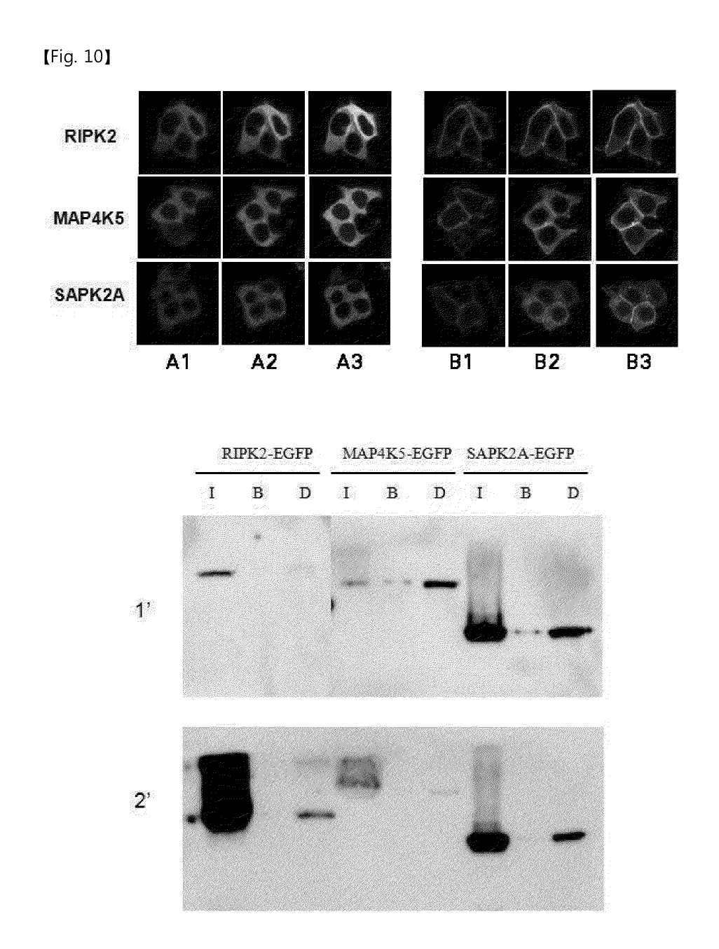

Referring to FIGS. 5, 6, 7, 8, 9, 10, and 12, the second construct dasatinib-biotin was introduced into a medium in which cells expressing the first construct and the second construct were grown, and then was allowed to infiltrate into the cells by culturing for 4 hours.

As a result, it can be seen that the fluorescence of the third construct (green) to which the prey binding to dasatinib was bound was translocated to the plasma membrane at the time of PMA treatment, like in the red fluorescence (the first construct) to which the translocation module was bound.

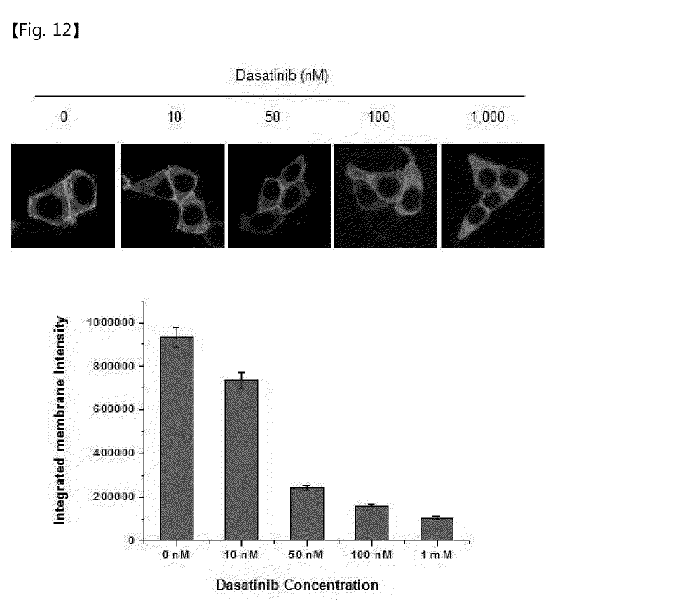

On the contrary, as for treatment with unbiotinylated dasatinib in its different concentrations, and as can be seen from the results shown in FIG. 12, the translocation of CSK protein (third construct, green) to the plasma membrane was inhibited by dasatinib in a concentration-dependent manner.

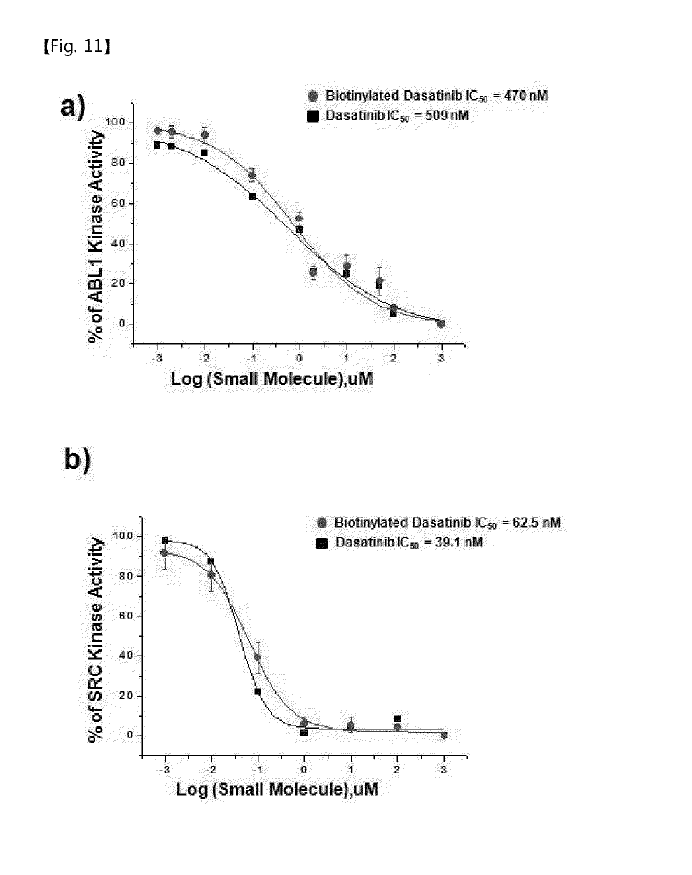

Referring to the lower panels of FIGS. 8, 9, and 10, where I: Input, B: biotin, and D: biotin-dasatinib, the proteins bound to dasatinib as a compound were confirmed through imaging analysis, and the binding of the respective proteins was again verified through immunoprecipitation (IP). As can be seen in FIG. 11, the phosphorylation activity of the biotinylated compound (dasatinib) was very similar to that of the original unbiotinylated compound. Therefore, it was confirmed that a prey protein can be identified under physiological conditions in which exemplary embodiments of the present invention do not affect the change in drug target activity.

### Example 5

### Verification of Other Compound-Protein Binding for Generalization

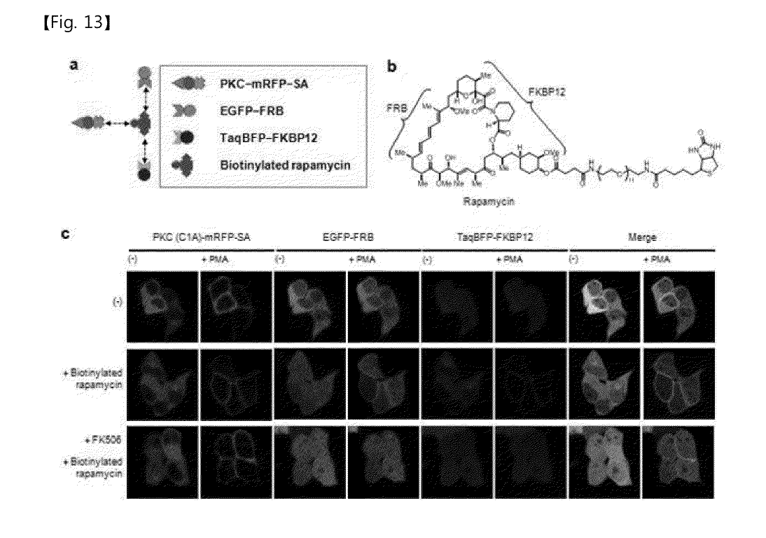

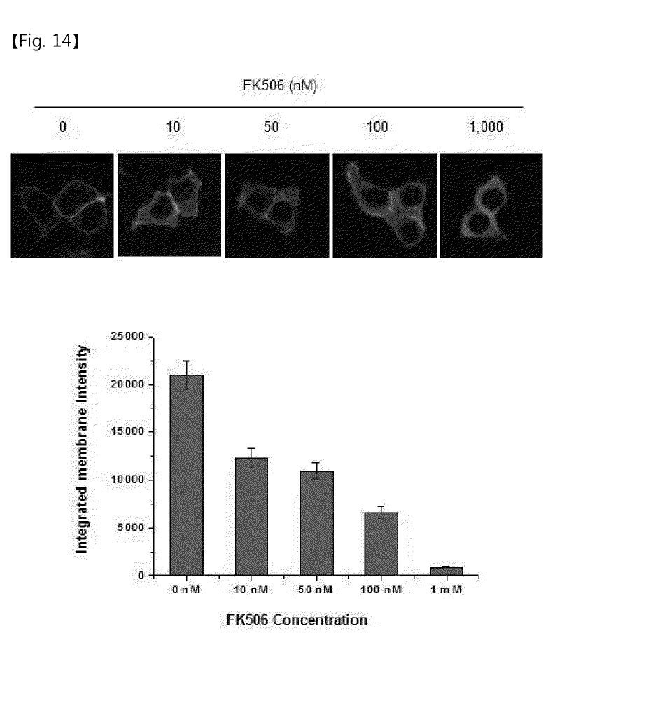

The above-described examples verified the effectiveness of exemplary embodiments the present invention. Also, the generalization of the methods was confirmed by using other compounds (see FIG. 13). When the biotin was bound to rapamycin as another anticancer drug, it was confirmed that FRB and FKBP12 proteins which have been known to bind with rapamycin were simultaneously detected, and the binding was inhibited by another compound FK506 which has been known as a competitive inhibitor against rapamycin. This verifies that the protein binding changes depending on the change in concentration of the competitive inhibitor, and thus the target protein FRB and FKBP12 specifically bind to rapamycin-biotin.

What is claimed is:

1. A method for screening a prey that interacts with a bait, the method comprising:
creating a cell that expresses:
a first construct, the first construct comprising a translocation module, a first labeling material, and a first medium; and
a third construct comprising a prey and a second labeling material;
introducing a second construct into the created cell, the second construct comprising a bait and a second medium configured to bind with the first medium;
reacting the prey and the bait; and
detecting intracellular distributions of the first construct and the third construct to determine an interaction between the prey and the bait.

2. The method of claim 1, wherein the reacting of the prey and the bait comprises treating the prey and bait with a signal material.

3. The method of claim 1, wherein the translocation module is selected from the group consisting of protein kinase C and variants thereof.

4. The method of claim 1, wherein the translocation module has an amino acid sequence selected from the group consisting of SEQ ID NO: 1, SEQ ID NO: 3, SEQ ID NO: 5, and SEQ ID NO: 7.

5. The method of claim 1, wherein the first labeling material is selected from the group consisting of green fluorescent protein (GFP), enhanced green fluorescent protein (EGFP), red fluorescent protein (RFP), monomeric red fluorescent protein (mRFP), Discosoma sp. red fluorescent protein (DsRed), cyan fluorescent protein (CFP), cyan green fluorescent protein (CGFP), yellow fluorescent protein (YFP), azami green (AzG), Heteractis crispa red fluorescent protein (HcR, HcRed), and blue fluorescent protein (BFP).

6. The method of claim 1, wherein the second labeling material is selected from the group consisting of green fluorescent protein (GFP), enhanced green fluorescent protein (EGFP), red fluorescent protein (RFP), monomeric red fluorescent protein (mRFP), Discosoma sp. red fluorescent protein (DsRed), cyan fluorescent protein (CFP), cyan green fluorescent protein (CGFP), yellow fluorescent protein (YFP), azami green (AzG), Heteractis crispa red fluorescent protein (HcR, HcRed), and blue fluorescent protein (BFP).

7. The method of claim 2, wherein the treatment with a signal material is performed using 50 nM to 5 uM of phorbol 12-myristate 13-acetate (PMA, phorbol ester).

8. The method of claim 1, wherein:
the first medium comprises streptavidin; and
the second medium comprises biotin.

9. The method of claim 1, wherein:
the first medium comprises dihydrofolate reductase (DHFR); and
the second medium comprises methotrexate (MTX).

10. A cell, comprising:
a first construct comprising a translocation module, a first labeling material, and a first medium; and
a third construct comprising a prey and a second labeling material.

11. The cell of claim 10, wherein the cell is selected from the group consisting of aCHO-k1 cell, HEK293 cell, HeLa cell, SH-SY5Y cell, Swiss 3T3 cell, 3T3-L1 cell, NIH/3T3 cell, L-929 cell, Rat2 cell, RBL-2H3 cell, and MDCK cell.

12. The cell of claim 10, wherein the first medium comprises streptavidin or dihydrofolate reductase (DHFR).

13. A screening kit, the kit comprising:
a cell comprising:
a first construct comprising a translocation module, a first labeling material, and a first medium, and
is a third construct comprising a prey and a second labeling material; and
a second construct comprising:
a second medium configured to bind with a first medium; and
a bait.

14. The kit of claim 13, wherein the first medium comprises streptavidin and the second medium comprises biotin.

15. The kit of claim 13, wherein:
the first medium comprises dihydrofolate reductase (DHFR); and
the second medium comprises methotrexate (MTX).

16. The kit of claim 13, further comprising a signal material that facilitates binding of the prey and the bait.

17. The method of claim 2, wherein the translocation module is selected from the group consisting of protein kinase C and variants thereof.

18. The method of claim 2, wherein the translocation module has an amino acid sequence selected from the group consisting of SEQ ID NO: 1, SEQ ID NO: 3, SEQ ID NO: 5, and SEQ ID NO: 7.

19. The method of claim 2, wherein the first labeling material is selected from a group consisting of green fluorescent protein (GFP), enhanced green fluorescent protein (EGFP), red fluorescent protein (RFP), monomeric red fluorescent protein (mRFP), Discosoma sp. red fluorescent protein (DsRed), cyan fluorescent protein (CFP), cyan green fluorescent protein (CGFP), yellow fluorescent protein (YFP), azami green (AzG), Heteractis crispa red fluorescent protein (HcR, HcRed), and blue fluorescent protein (BFP).

20. The method of claim 2, wherein the second labeling material is selected from the group consisting of green fluorescent protein (GFP), enhanced green fluorescent protein (EGFP), red fluorescent protein (RFP), monomeric red fluorescent protein (mRFP), Discosoma sp. red fluorescent protein (DsRed), cyan fluorescent protein (CFP), cyan green fluorescent protein (CGFP), yellow fluorescent protein (YFP), azami green (AzG), Heteractis crispa red fluorescent protein (HcR, HcRed), and blue fluorescent protein (BFP).

21. The method of claim 2, wherein:
the first medium comprises streptavidin; and
the second medium comprises biotin.

22. The method of claim 2, wherein:
the first medium comprises dihydrofolate reductase (DHFR); and
the second medium comprises methotrexate (MTX).
